(19) United States
(12) Patent Application Publication
(10) Pub. No.: US 2025/0140405 A1
(43) Pub. Date: May 1, 2025

(54) NETWORKED SYSTEM CONFIGURED TO IMPROVE ACCURACY OF HEALTH EVENT DIAGNOSIS

(71) Applicant: Medtronic, Inc., Minneapolis, MN (US)

(72) Inventors: Kevin T. Ousdigian, Shoreview, MN (US); Joel R. Lauer, Clearwater, MN (US); Amruta Paritosh Dixit, Maple Grove, MN (US); Shantanu Sarkar, Roseville, MN (US)

(21) Appl. No.: 18/693,823

(22) PCT Filed: Nov. 3, 2022
(86) PCT No.: PCT/IB2022/060592

§ 371 Date: Mar. 20, 2024

Related U.S. Application Data
(60) Provisional application No. 63/264,317, filed on Nov. 19, 2021.

(51) Int. Cl. G16H 50/20; G16H 10/60; G16H 40/67

(52) U.S. Cl. CPC G16H 50/20; G16H 10/60; G16H 40/67

(57) ABSTRACT

This disclosure is directed to systems and techniques configured to apply at least one criterion to health event data stored in a record for a patient for determining whether to remove at least a portion of the health event data from the record or retain that portion as an accurate reflection of patient health for that point-in-time. The health event data includes adjudicated health events and non-adjudicated health events over a first time period. Based on a determination that the health event data satisfies the at least one criterion, the example technique may direct the example system to remove the health event data corresponding to the adjudicated health events and the non-adjudicated health events from the record and then, adjust longitudinal diagnostic information of a second time period that includes the first time period.

### FIELD

The disclosure relates generally to medical systems and, more particularly, medical systems configured to manage resource consumption for monitoring patient activity to record data that accurately represents events in patient health.

### BACKGROUND

Medical systems may monitor various types of data of a patient or a group of patients for one or several purposes. Amongst the numerous examples, some medical systems may record measurements of a patient and their heart as indicia of cardiac health for that patient, which may be memorialized in raw data and/or processed data formats; as one example, electric signals representing cardiac activity over a period of time may be memorialized as a cardiac electrogram (EGM), and then processed into other indicia of the cardiac health of the patient. In some examples, the medical system may monitor the cardiac EGM to detect one or more types of arrhythmia, such as bradycardia, tachycardia, fibrillation, or asystole (e.g., caused by sinus pause or AV block).

Medical professionals may use the medical system for a number of reasons, such as having the medical system record patient data for future use. For various purposes, a medical professional may program the medical system to operate as desired, which may be in accordance with a certain algorithm, and calibrate medical system components to detect health events, deliver a therapy, and so forth. In some examples, the medical system may include one or more of an implantable medical device or a wearable device to collect various measurements used to detect changes in patient cardiac health. In some examples, an implantable or a wearable medical device may be configured with an algorithm for monitoring patient cardiac activity for cardiac events.

### SUMMARY

Medical systems and techniques as described herein detect changes in health for a patient based upon a variety of patient inputs. In general, the patient benefits from having a medical device that can use the patient inputs to detect such patient health changes with a reasonable degree of accuracy. The medical device may be equipped with programming for evaluating the patient inputs to determine an occurrence of a health event. Over time, the medical device may improve upon its accuracy and/or receive updates to its programming. However, false detections are still possible even though a medical device manufacturer and/or developer routinely updates the medical device and introduce new medical devices as replacements for a previous device. The medical system may implement one or more solutions for identifying these false detections.

If needed, the medical device of the patient may employ an algorithm (e.g., in its programming) to detect, as a health event, a change in patient health (e.g., cardiac health) caused by a specific malady (e.g., cardiac malady). While the algorithm may be configured to distinguish between patient data indicative of a true health event and patient data that is not a health event, each falsely detected health event could negatively affect the patient's medical care.

To illustrate by way of example, when the medical device first registers an initial detection of a health event that is then rejected as a false health event (or false detection), the initial detection has a negative impact on the information gleaned from the patient data during the inappropriate initial detection. Any clinician who desires to examine the patient data recorded during the initial detection of a true health event is not provided with an accurate patient history related to that health event. The clinician either cannot render an accurate conclusion or has to account for such inaccuracy in its conclusion. By removing data associated with the false health event, the medical system improves the quality of diagnostic data provided to clinicians and other users.

The present disclosure describes medical systems and techniques to filter (e.g., delete) patient data associated with a falsely-detected health event, thereby improving the quality of diagnostic data. While identifying false health events and removing their initial detection from a patient record improves the accuracy of the record, the medical device may have recorded/computed other information corresponding to the false health events. One example of such information is longitudinal data. Some medical systems and techniques achieve maximum coverage in its correction of the negatively affected longitudinal data. Some medical systems and techniques identify other health events that are false or inappropriate. Having more accurate patient histories facilitates streamlining and efficiency in actionable workflows of the clinician including streamlined health event review and reporting.

In one example, a medical system comprises an implantable medical device comprising: sensing circuitry configured to sense patient activity including one or more of a cardiac electrical signal, impedance, or motion of a patient; communication circuitry configured to communicate with a remote computing device; and processing circuitry configured to: analyze the sensed patient activity; detect health events of the patient based on the analysis; and transmit health event data to the remote computing device for the detected health events; and the remote computing device comprising: processing circuitry; and memory comprising programming instructions that, when executed by the processing circuitry, cause the processing circuitry to: store the health event data received from the implantable medical device in a record for the patient; apply at least one criterion to the health event data stored in the record for the patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, remove the health event data corresponding to the adjudicated health events and the non-adjudicated health events from the record; adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

In another example, a method performed by a medical system comprises: applying, by a remote computing device of the medical system, at least one criterion to health event data stored in a record for a patient for determining whether to remove at least a portion of the health event data from the record, wherein an implanted medical device of the patient communicates the health event data to the remote computing device, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, remove the health event data corresponding to the adjudicated health events and the non-adjudicated health events from the record; adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

In another example, a non-transitory computer readable storage medium comprising program instructions configured to cause processing circuitry to perform steps comprising: applying at least one criterion to health event data stored in a record for a patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, removing the health event data corresponding to the adjudicated health events and the non-adjudicated health events from the record; adjusting longitudinal diagnostic information of a second time period that includes the first time period based on removing the adjudicated health events and the non-adjudicated health events from longitudinal diagnostic information of the time period; and generating output data indicative of the modified longitudinal diagnostic information of the second time period.

In yet another example, a medical system comprises processing circuitry; and memory comprising programming instructions that, when executed by the processing circuitry, cause the processing circuitry to: apply at least one criterion to the health event data stored in a record for a patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, remove the health event data corresponding to the adjudicated health events and the non-adjudicated health events from the record; adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a functional block diagram illustrating an example configuration of a medical device, in accordance with one or more examples of the present disclosure.

FIG. 3 is a conceptual side-view diagram illustrating an example configuration of the IMD of FIGS. 1 and 2, in accordance with one or more examples of the present disclosure.

FIG. 4 is a functional block diagram illustrating an example configuration of the external device of FIG. 1, in accordance with one or more examples of the present disclosure.

FIG. 5 is a block diagram illustrating an example system that includes an access point, a network, external computing devices, such as a server, and one or more other computing devices, which may be coupled to the medical device and external device of FIGS. 1-4, in accordance with one or more examples of the present disclosure.

FIG. 6 is a flow diagram illustrating an example operation for adjusting patient data to reflect accurate detection of changes in patient health, in accordance with one or more examples of the present disclosure.

FIG. 7 is a block diagram illustrating an example flow via the medical system of FIG. 1 to provide accurate health event data for clinician review after adjudication and adjustment, in accordance with one or more examples of the present disclosure.

FIGS. 8A-8B are each a representation of example output for clinician review from the example flow of FIG. 7, in accordance with one or more examples of the present disclosure.

Like reference characters denote like elements throughout the description and figures.

### DETAILED DESCRIPTION

In general, medical systems according to this disclosure implement techniques for detecting changes in patient health. A patient may have a medical device of the system implanted into their body for long-term monitoring, diagnostics, and detection. To that end, the medical device may employ hardware/software components that are programmed for capturing, in memory, one or more samples of various patient data corresponding to a point-in-time. The medical device may further employ logic that when executed, evaluates the one or more samples of captured patient data for sufficient indicia of a health event. There are number of techniques that may be implemented as the logic in the medical device and the example operation represents at least a portion of those techniques. Example medical devices that may collect patient data may include an implantable or wearable monitoring device, a pacemaker/defibrillator, a ventricular assist device (VAD), or a neurostimulator.

The example medical device may communicate the patient data to other devices, such as a computing device, and those devices may further analyze the patient data and then, provide a report regarding the patient's activities and health. The report may compare various implementations of the techniques described herein, for example, comparing, for the same patient, health event data with adjudication/adjustment and health event data without adjudication/adjustment. The report may provide a patient, clinician, or caregiver information an important aspect of the patient's health. While not every health event can be adjudicated, by adjudicating a fraction, the medical system can determine whether there is any benefit to storing any health event data for that day (or other time period).

In this manner, the techniques of this disclosure may advantageously enable improved accuracy in the detection of changes in patient health and, consequently, better evaluation of the condition of the patient.

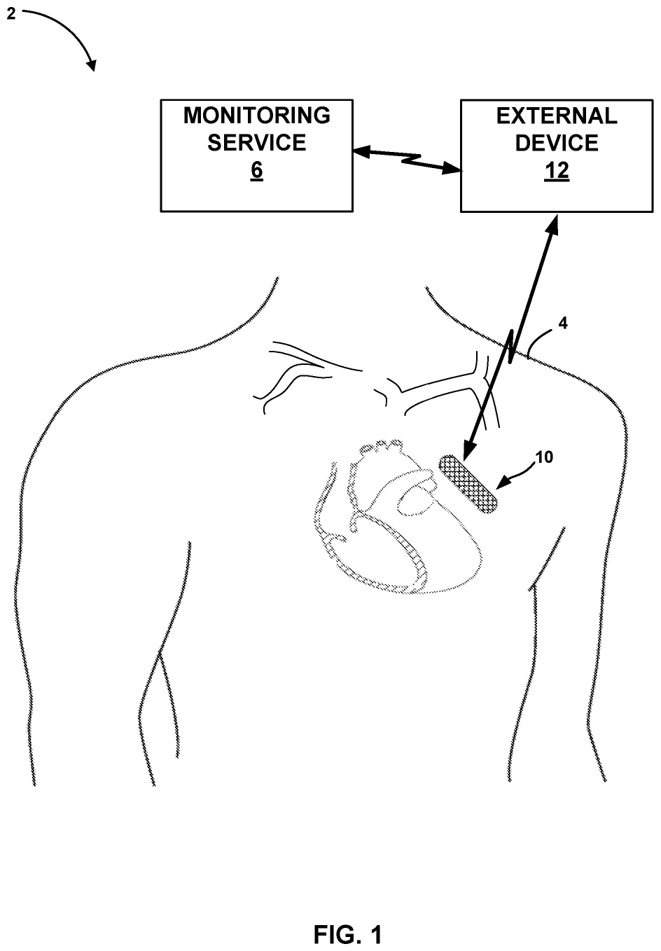

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM available from Medtronic, Inc. of Minneapolis, MN.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10.

External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

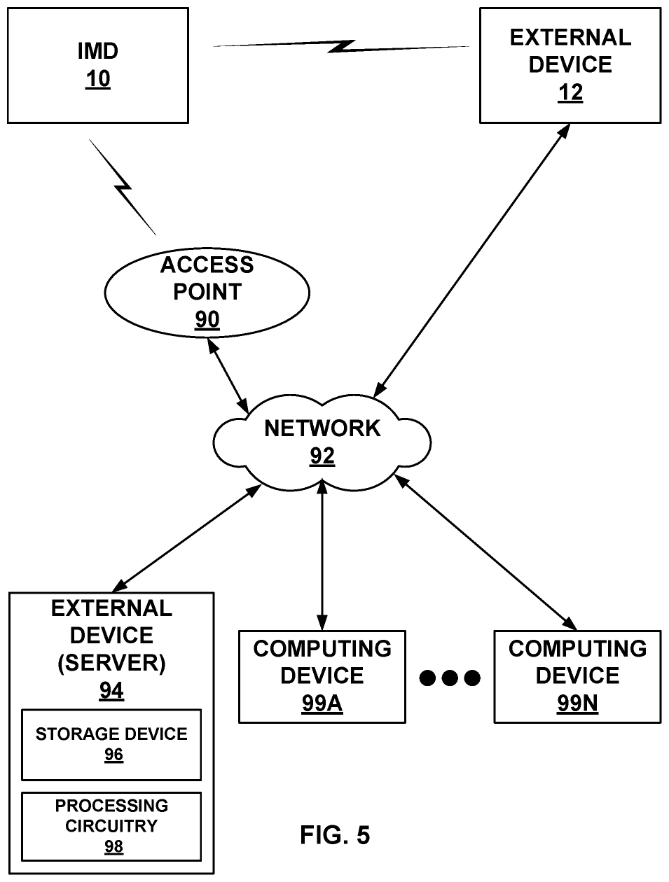

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve cardiac EGM segments recorded by IMD 10 due to IMD 10 determining that an episode of asystole or another malady occurred during the segment. As another example, external device 12 may receive activity data, daily activity metric values, or other data related to the techniques described herein from IMD 10. As will be discussed in greater detail below with respect to FIG. 5, one or more remote computing devices may interact with IMD 10 in a manner similar to external device 12, e.g., to program IMD 10 and/or retrieve data from IMD 10, via a network.

Monitoring service 6 refers to a computing service, which may be similar to that provided by the Medtronic CareLink® Network, that communicates with IMD 10 directly over a network connection and/or indirectly through external device 12. One or more aspects of medical system 2 of FIG. 1 may be implemented with networking infrastructure connecting computing devices of monitoring service 6 with IMD 10 and/or external device 12. As described herein, uploads of various datasets (e.g., health event data, samples of patient data, longitudinal diagnostic information, and/or the like) may trigger monitoring service 6 into adjudicating initial detections by IMD 10 of health events (e.g., cardiac events) and if needed, adjusting IMD 10's device history of IMD 10 to more accurately reflect patient 4's health.

Processing circuitry of medical system 2, e.g., of one or more devices of monitoring service 6, IMD 10, external device 12, and/or of one or more other computing devices, may be configured to perform the techniques described herein. According to some example techniques, while monitoring patient health and detecting any changes in patient health (e.g., health events), medical system 2 of this disclosure may be directed to manage memory resources at a medical device, for example, by only retaining, in those memory resources, a record that accurately represents the monitored patient activity/detected changes in patient health. The processing circuitry of medical system 2 may employ various known mechanisms to capture (e.g., samples) of various patient data over a period of time, analyze the captured physiological parameter values for indicia of any health events including non-trivial changes in patient health, and then, determine whether to remove the captured patient data for the health events from the record. In some examples, the processing circuitry of medical system 2 analyzes a cardiac EGM or ECG and other patient activities sensed by IMD 10 and may identify indicia of a cardiac episode, such as an arrhythmia, or another cardiac event that either has occurred or is occurring in patient 4.

Processing circuitry of the one or more devices of monitoring service 6, may apply at least one criterion to health event data stored in a record for a patient for determining whether to remove at least a portion of the health event data from the record. The record may be a database record stored in a database system for monitoring service 6, IMD 10, external device 12, and/or one or more other devices. The health event data in the record may include one or more adjudicated health events and one or more non-adjudicated health events over a first time period. For each adjudicated health event, the health event data may indicate a true detection or a false detection by IMD 10 or another medical device of monitoring service 6. After adjudicating each health event that qualifies for adjudication, the health event data may be indicative of one or more health events adjudicated as true detections of the health event and/or one or more health events adjudicated as false detections. Various criterion may be programmed into (e.g., adjustment logic of) the one or more devices of monitoring service 6 for comparing with the health event data to perform an adjustment of the health event data.

Based on a determination that the health event data satisfies at least one criterion, the processing circuitry removes the health event data corresponding to the adjudicated health events and the non-adjudicated health events from the record. By doing so, the processing circuitry reduces the memory capacity being consumed to store the record, which includes inaccurate health event data. This may benefit IMD 10 and other medical devices that are restricted resource-wise, for example, with a smaller resource footprint by freeing memory resources for accurate health event data. Implanted medical devices with a limited memory capacity can retain only accurate health event data to reserve critical memory resources.

As an option, the processing circuitry of the one or more devices of monitoring service 6 may reprogram (e.g., detection logic of) medical devices communicatively coupled to the one or more devices of monitoring service 6. The processing circuitry may communicate a message to modify, add, and/or remove settings information (e.g., detection settings). In general, each setting may refer to a parameter used in determining whether time-stamped data recording electrical activity of patient 4's heart. The processing circuitry of the one or more devices of monitoring service 6 may select the reprogramming option, upon determining satisfaction of the at least one criterion or at a later date, submit the message to reduce future occurrences of false detections. Based on data from a previous adjudication, the processing circuitry may generate new setting information by adjusting values for various sensing and detection parameters to prevent a same or similar type of false detection. For example, if IMD 10 detects a health event that is later adjudicated as a false detection and that false detection is classified as due to T-wave oversensing, the processing circuitry may modify current setting information of IMD 10 to reduce a likelihood of detecting another health event due to T-wave oversensing. The processing circuitry may communicate a message to IMD 10 instructing a (client) component to modify a blanking or sensing threshold decay delay. Alternatively, the processing circuitry may determine that the false detection is classified as being due to myopotential noise and automatically communicate a message changing one or more noise detection logic parameters. As yet another example, the processing circuitry may determine that the false detection results from sinus tachycardia and as a response, automatically communicate a message directing IMD 10 to change algorithm detection parameters for Atrial Fibrillation (AF) od Tachycardia detection logic. Similarly, the processing circuitry of the one or more devices of monitoring service 6 may generate new setting information for PVC or QT detection logic.

As another option, the processing circuitry of the one or more devices of monitoring service 6 may modify any one or more of the at least one criterion used to remove the health event data corresponding to the adjudicated health events from the record.

Because some medical devices support a clinician of patient 4 with various services related to patient data, removing false detections of both adjudicated and non-adjudicated health events as described above allows monitoring service 6 to provide (for the most part) only accurate health event data to the clinician. The processing circuitry may use the accurate health event data to generate additional information that accurately describes the health (e.g., cardiac health) of patient 4. For instance, the processing circuitry may generate longitudinal diagnostic information to describe patient 4's health of which some examples include a counter (e.g., an arrhythmia counter), a duration (e.g., an arrhythmia duration), an alert (e.g., an arrhythmia alert), sensed patient data (e.g., ECG data), a trend, a histogram and/or the like. The longitudinal diagnostic information may correspond to a second time period, which is longer than and encompasses the first time period. In some examples, one of or both the first time period and the second time period may be modified by patient 4, patient 4's clinician, or another user/caregiver. This information may form part of a history of patient 4's health events as well as a device diagnostic history. Hence, preserving true detections of health events enables the processing circuitry to generate the longitudinal diagnostic information from primarily accurate records of patient 4's health history.

The processing circuitry of the one or more devices of monitoring service 6 may adjusts the longitudinal diagnostic information of the second time period that includes the first time period based on removing the adjudicated health events and the non-adjudicated health events from longitudinal diagnostic information of the time period. The processing circuitry may generate output data indicative of the modified longitudinal diagnostic information of the second time period.

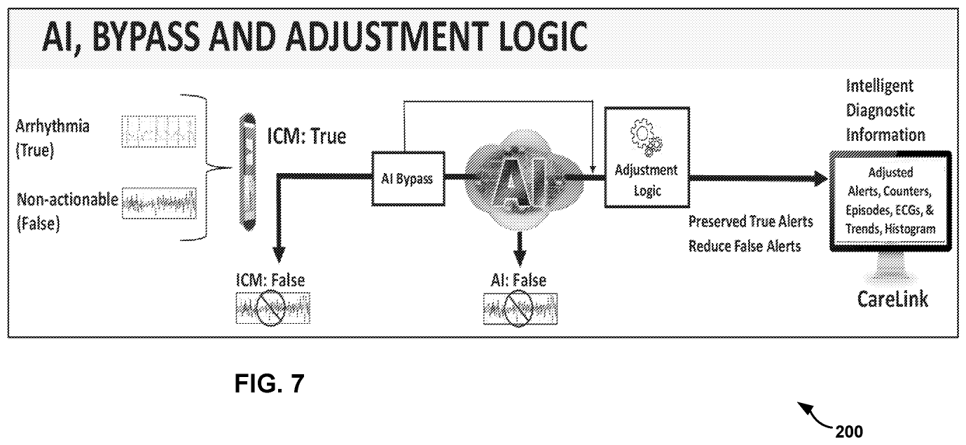

Processing circuitry of IMD 10 may be communicably coupled to one or more sensors, each being configured to sense patient data (e.g., physiological parameters) in some form, and sensing circuitry configured to generate sensor data and other patient data. Processing circuitry of medical system 2, such as processing circuitry of IMD 10 and/or processing circuitry of external device 12, may compute values representing some aspect of patient 4's physiology at a particular point-in-time and the computation of these values may be in accordance with a number of applicable metrics and other mechanisms for computing such patient data. As described herein, processing circuitry of IMD 10, possibly in combination with processing circuitry of external device 12, may employ various techniques to capture the above patient data over a period of time, analyze the captured physiological parameter values for indicia of any health events including cardiac episodes and non-trivial changes in patient 4's cardiac health, and then, determine whether to remove or retain the captured physiological parameter values from a record for that time period. As explained in detail for FIG. 7, the above techniques correspond to an initial detection analysis, within a medical device such as an implant, which may be followed by an adjudication and if needed, an adjustment of inaccuracies in device and/or patient history including longitudinal diagnostic information.

The present disclosure envisions medical devices that are equipped with a number of hardware/software components to implement different example techniques. IMD 10, as one medical device, may be configured with detection logic to implement techniques to determine whether patient 4 is experiencing/has experienced/will (imminently) experience an example cardiac episode. As described in the present disclosure, the detection logic may employ a number of compatible mechanisms to successfully implement the above techniques, such as a machine learning model and/or a decision tree, where each mechanism prescribes criterion that the detection logic may use for distinguishing patient data indicative of a true cardiac episode from patient data that does not indicate a true cardiac episode (i.e., a false cardiac episode).

IMD 10 may offload part of the detection logic to external device 12 or one or more devices of monitoring service 6. Memory uplink interrogation refers to a protocol by which IMD 10 may offload one or more samples of patient data for remote evaluation via a machine learning model, according to one example where offloading may be used in an adjudication and/or adjustment technique. Because IMD 10 may use the memory uplink interrogation protocol for uploading a device history describing various health event data (including longitudinal diagnostic information), external device 12 or the one or more devices of monitoring service 6 may correct inaccuracies in the device history. Alternatively, external device 12 or the one or more devices of monitoring service 6 may return adjudication results to IMD 10 for use in correcting inaccuracies in the device history.

IMD 10 and other implanted medical devices described herein have a number of restrictions such as size limitations due to being required to fit within the human body and inherent resource limitations inherent to implanted medical devices in general; to overcome/mitigate these restrictions, IMD 10 may benefit from offloading adjudication and/or adjustment (especially when availing the memory uplink interrogation protocol). If offloading is not available, IMD 10, as an option, may manage its own memory resources to retain (for the most part) a device history reflecting only true detections.

In any device, an adjustment technique may be accomplished in intervals, for example, by removing health event data corresponding to false detections and preserving health event data corresponding to true detections occurring over a first time period (e.g., one day). Based on the removed and/or preserved health event data, the adjustment technique may update the longitudinal diagnostic information recorded for a longer second time period (e.g., a week) that includes the first time period.

Although described in the context of examples in which IMD 10 is a cardiac device that senses a cardiac EGM or ECG to detect cardiac events, such as arrhythmias, example systems including one or more implantable, wearable, or external devices of any type configured to identify health events may be configured to implement the techniques of this disclosure.

In some examples, processing circuitry in a wearable device may execute same or similar logic as the logic executed by processing circuitry of IMD 10 and/or other processing circuitry as described herein. In this manner, a wearable device or other device may perform some or all of the techniques described herein in the same manner described herein with respect to IMD 10. In some examples, the wearable device operates with IMD 10 and/or external device 12 as potential providers of computing/storage resources and sensors for monitoring patient activity and other patient parameters. For example, the wearable device may communicate the patient data to external device 12 for storage in non-volatile memory and for applying metric and computing parameter values from sensed patient activity.

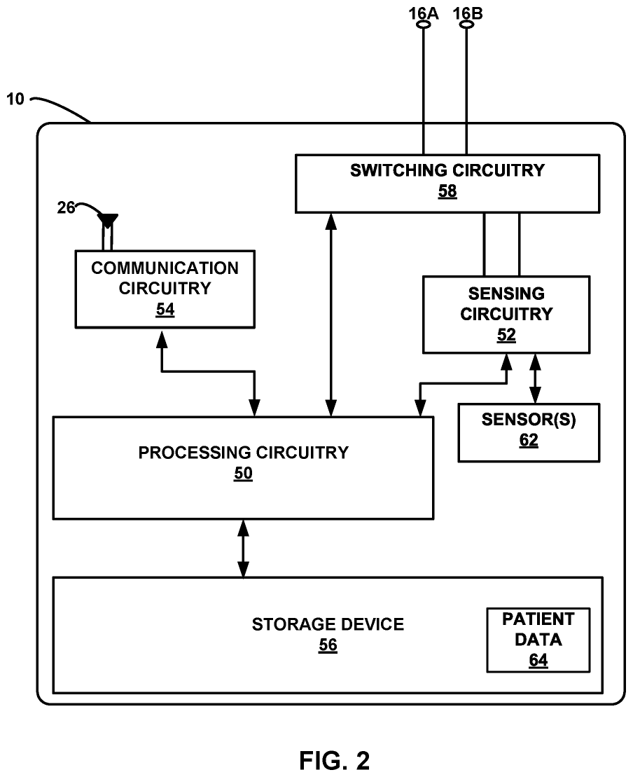

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical signals of the heart of patient 4, for example by selecting the electrodes 16 and polarity, referred to as the sensing vector, used to sense a cardiac EGM or ECG, as controlled by processing circuitry 50. Sensing circuitry 52 may sense signals from electrodes 16, e.g., to produce a cardiac EGM or ECG, in order to facilitate monitoring the electrical activity of the heart (e.g., cardiac activity). Sensing circuitry 52 may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62. Sensing circuitry 52 may capture signals from any one of electrodes 16 and sensors 62, e.g., to produce patient data, in order to facilitate (e.g., remote) monitoring (e.g., by an external computing system) and detecting changes in patient health.

Sensing circuitry 52 may generate patient data from the captured signals that encode sensed patient activity, including the patient's cardiac activity as described herein. Sensing circuitry 52 and processing circuitry 50 may store patient data in storage device 56. Patient data may include segments, e.g., a time sequence of samples of cardiac EGM data or ECG data, for example. Patient data may include various values determined from the captured signals, such as heart rate, heart rate variability, or morphological values. Processing circuitry 50 may detect events or episodes, e.g., of arrhythmia, based on the captured signals or patient data derived therefrom, and store indications of the detection of such events as patient data. Patient data may include signal segments and values corresponding to the occurrence of the detected event. Various metrics enable standardized measurement of each sample (e.g., timestamp) of physiological parameter data and differentiation between multiple samples (e.g., timestamps or longer time periods and/or patients) of physiological parameter data. Patient data may be uploaded to an external device, such as external device 12 of FIG. 1, or over a network to a computing service, such as monitoring service 6 of FIG. 1.

Processing circuitry 50 may control one or more of sensors 62 to sense the above patient data in some form (e.g., patient activity); examples of one or more sensors 62 to sense patient activity include an accelerometer (e.g., a three-axis accelerometer), a gyroscope, a temperature gauge, a moment transducer, and/or the like. There are a number of methods for converting the patient activity data into one or more physiological parameters, each of which may be a quality (e.g., high activity, low activity, and/or the like) or a quantity (e.g., a number of activity minutes (e.g., 10-second blocks)), representing some aspect of the patient's physiology.

Processing circuitry 50, executing logic configured to perform a detection analysis on the sensor data, is operative to detect any change (e.g., a decline) in patient health. Processing circuitry 50, executing detection logic configured to perform a detection analysis for cardiac episodes, including arrhythmias, that are likely to cause a change (e.g., a decline) in patient health or, otherwise, negatively affect the patient's heart. Processing circuitry 50 may control sensing circuitry 52 to sense cardiac physiology of the patient in some form, e.g., by sensing electrical activity of the patient's heart via a plurality of electrodes, such as electrodes 16. As described herein, the detection analysis is performed on cardiac electrogram (EGM) data and any other sensor data, in the context of FIG. 2, corresponding to the sensed electrical activity stored as patient data 64.

Sensing circuitry 52 converts to digital form signals corresponding to the sensed electrical activity of an electrocardiogram (ECG) and provides the digitized signals to processing circuitry 50 for the detection analysis. A “wave” or “waveform” refers to a type of ECG-component and each component type may be represented in corresponding cardiac EGM data. It should be noted that the cardiac EGM data for a typical ECG records a series of points on waves (e.g., the P wave, Q wave, R wave, S wave, T wave and U wave), intervals (e.g., PR interval, QRS interval (also called QRS duration), QT interval or RR interval), segments (e.g., PR segment, ST segment or TP segment), complex(es) (e.g., QRS complex), and other components. In some examples, the cardiac EGM for any of these ECG-components may be arranged into one or more vectors of data values that represent the sensed electrical activity. In this example, the ECG refers to a combination of the cardiac EGM data provided by respective pairs of electrodes connected to the patient's heart; each respective dataset of the cardiac EGM data is a vector of data values representing at least a portion of the sensed electrical activity, such as any one or more of the above ECG components.

For an example ECG representing sensed electrical activity encompassing a period of time (e.g., a minute, a day, a month, as so forth) during which a cardiac event may have occurred, patient data 64 stores vectors of data values representative of a cardiac rhythm in which, in some examples, an example vector includes a sequence of values representing a particular waveform of the cardiac rhythm. Processing circuitry 50 may apply a pattern recognition technique to interpret the above vectors of data values. In some examples, a particular waveform (e.g., R wave) may indicate an initial detection of a cardiac event. In patient data 64 of IMD 10, storage device 56 may use health event data to record the initial detection of the cardiac event, for example, in longitudinal diagnostic information (e.g., by incrementing a cardiac event counter and/or incorporating a duration of the cardiac event into an average duration parameter). Processing circuitry 50 may select, based on the cardiac event, the particular waveform for retention (e.g., and subsequent memory uplink interrogation). The cardiac EGM data for the particular waveform may be stored as a sample where the example ECG records the initial detection of cardiac event and is further representative of a totality of ECGs being monitored by IMD 10. Processing circuitry 50 may store one or more data value vectors corresponding to the particular waveform as at least part of the sample t. As described herein, the sample may be (e.g., uploaded to a cloud service such as monitoring service 6 and) used to confirm the cardiac event, completing the detection analysis according to some examples.

Processing circuitry 50 may be further configured to apply a machine learning model to one or more samples of the above vectors over a time period, and based on prediction values of the machine learning model, determine whether the one or more samples indicates a likely occurrence of a cardiac event. The machine learning model may be used as part of the initial detection analysis or used to confirm an initial detection. Processing circuitry 50 generates for display output data indicative of an initial detection of a cardiac event and/or a confirmation of an initial detection of a cardiac event.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include the patient data described above including any health event data as described herein for FIG. 1. In general, the health event data identifies occurrences of cardiac arrhythmia and/or indications of changes in patient health including indications of true detections.

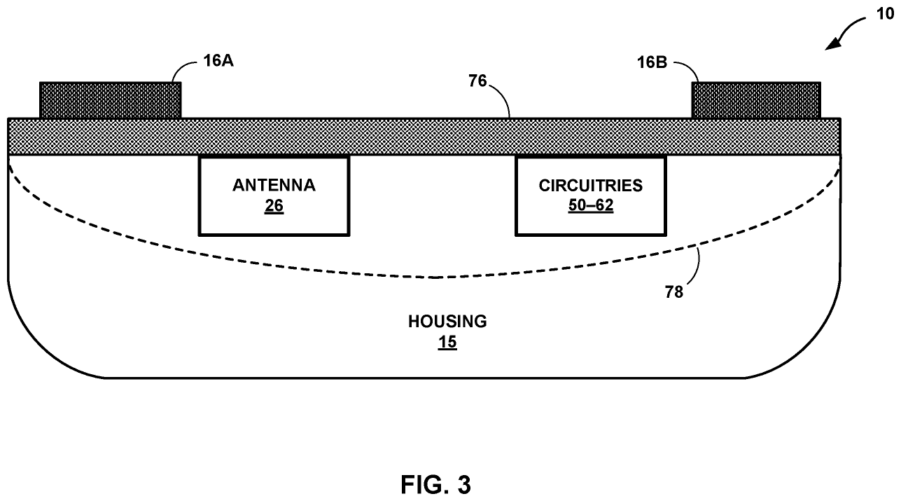

FIG. 3 is a conceptual side-view diagram illustrating an example configuration of IMD 10 of FIGS. 1 and 2. While different examples of IMD 10 may include leads, in the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

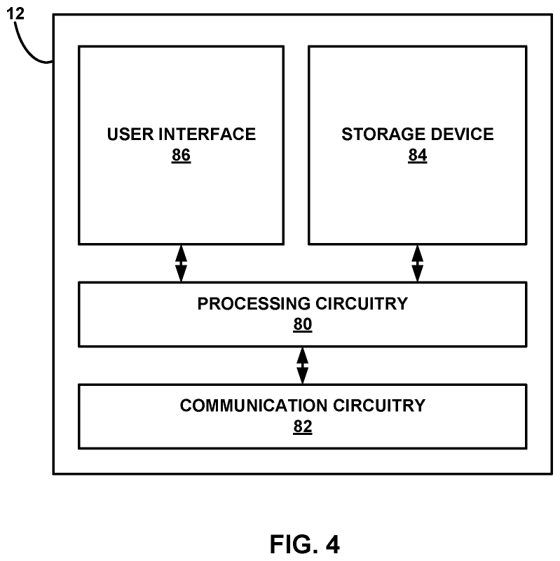

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., asystole episode data) to external device 12. In turn, external device 12 may receive the collected data from IMD 10 and store the collected data in storage device 84. The data external device 12 receives from IMD 10 may include episode data (e.g., cardiac EGMs), parameters, patient activity, sensor data 64. Processing circuitry 80 may implement any of the techniques described herein to analyze data 64 from IMD 10 to determine daily activity metric values e.g., to determine whether the patient is experiencing a change in health e.g., based upon one or more criteria.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., daily activity metric values, indications of changes in daily activity metric values, and indications of changes in patient health that correlated to the changed in daily activity metric values, determinations of probability data of possible medical conditions to predict, sensor data, physiological parameters, metric values, episode data, cardiac EGM, ECG, electrocardiogram, cardiac electrogram. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 5 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as a patient's medical device history including indications of cardiac arrhythmia episodes and other cardiac events, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink® Network. In some examples, one or more aspects of the example system of FIG. 5, e.g., server 94, may implement monitoring service 6.

In some examples, one or more of computing devices 100 may be a tablet or other smart device located with a clinician, by which the clinician may program, receive alerts from, and/or interrogate IMD 10. For example, the clinician may access data, such as patient data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician.

Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. 5 computing devices 99 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 99 may implement any of the techniques described herein to analyze information, data, or data received from IMD 10, e.g., to determine whether the health status of a patient has changed, to determine whether adjudication criteria are satisfied and/or adjustment criteria are satisfied.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

In storage device 96, a database system may organize records of health event data. In one example, a first structure of the health event data include metadata for identifying any one of the non-adjudicated health events and a second structure of the health event data includes for any one of the adjudicated events, metadata for identifying that non-adjudicated health event and a sample of sensed patient data. In one example, a third structure of the health event data include for any one of a number of unqualified health events, metadata for identifying that unqualified health event, a sample of sensed patient data captured during that unqualified health event, and a reason for failing to qualify for adjudication (e.g., disqualification).

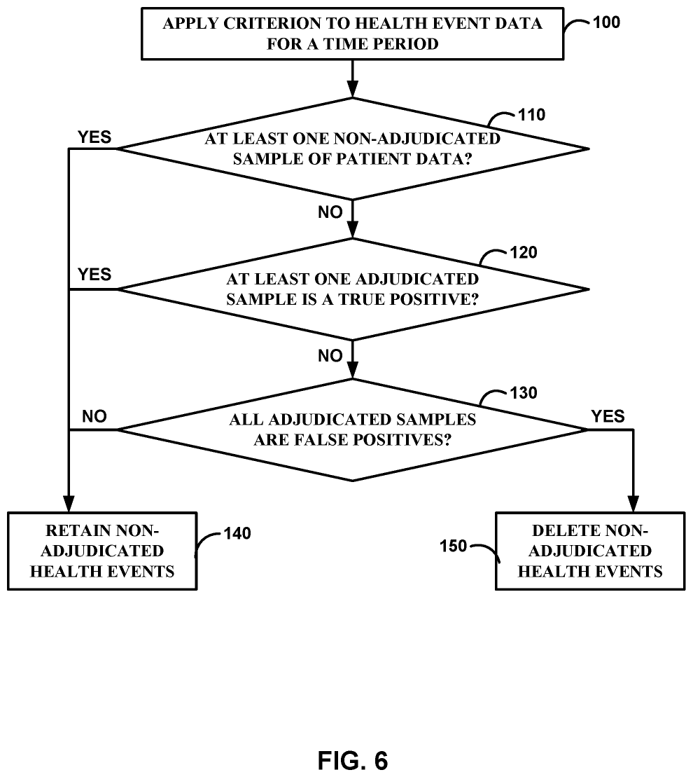

FIG. 6 is a flow diagram illustrating an example operation for enabling memory resource capacity conservation in a medical device while reserving the memory resource capacity for information reflecting accurate detection of changes in patient health, in accordance with one or more examples of the present disclosure. In some examples, the example operation may be implemented in a variety of embodiments of medical system 2 of FIG. 1. One or more devices described in FIGS. 1-5 may perform the example operation while communicatively coupled to the medical device where health event data is stored in memory. Device(s) operating a computing service over a network (e.g., monitoring service 6), a patient device such as a mobile device (e.g., external device 12), and/or the medical device itself may perform the example operation.

According to the illustrated example operation of FIG. 6, a patient may have the above-mentioned medical device implanted into their body for long-term monitoring, diagnostics, and detection. To that end, the medical device may employ hardware/software components that are programmed for capturing, in memory, one or more samples of various patient data corresponding to a point-in-time. Patient data as described herein may refer to data generated from any sensed signal(s) from a device component or captured by an external sensor. The medical device may further employ logic that when executed, evaluates the one or more samples of captured patient data for sufficient indicia of a health event. There are number of techniques that may be implemented as the logic in the medical device and the example operation represents at least a portion of those techniques.

Within a (server) computing device operated by monitoring service 6, processing circuitry executes logic for implementing the following steps of the example operation. IMD 10, as an example of the above medical device, provides input for the logic including a dataset of health event data (e.g., cardiac event data) corresponding to a particular time period (e.g., 1 day) and a certain number of samples of patient data (e.g., 4 segments of ECG readings).

The health event data may be generated and/or stored within IMD 10 as part of an initial detection analysis for possible health events. In one example initial detection analysis, processing circuitry 50 of IMD 10 monitors patient data generated by sensing circuitry 52 of IMD 10 and stored in memory of storage device 56 and then, determines whether a sample indicates a likely occurrence of a health event. Results of the initial detection analysis-including a count of the possible health events and an average duration for each health event-may be stored as a portion of the health event data in memory of storage device 56. The present disclosure describes a number of mechanisms by which processing circuitry 50, at a later point in time, may transmit, via communication circuitry 54 of IMD 10, the health event data from the memory in the storage device to another device for performance of the example operation.

In the example operation of FIG. 6, processing circuitry within a (server) computing device operated by monitoring service 6 applies at least three criterion to health event data for a time period (100). It should be noted that while FIG. 6 illustrates three (3) decision blocks, other examples of the example operation of FIG. 6 include only one (1) decision block representing only one (1) criterion. As described herein, the health event data refers to a portion of IMD 10's device history and as such, generally describes an aspect of patient health that is subject to change through that portion of the device history. Each criterion of FIG, 6 enables the server computing device of monitoring service 6 to identify potential errors in the initial detection analysis, for example, resulting in false detections of health events (i.e., adjudication).

An application of the criterion of FIG. 6 functions as a mitigation mechanism for resource limitations inherent to implanted medical devices such as IMD 10. To illustrate, although a considerable number of health events may be recorded over the first time period, memory restrictions within IMD 10 permit storage of patient data samples for only a fraction of those recorded health events; none of the remaining initial detections of health events can be adjudicated. Without sampled patient data for significantly more (if not all) initial detections of health events, a health event that should have been adjudicated as a false detection is treated as a true detection, and since any data associated with that health event is inaccurate, the entire device history is rendered inaccurate. For example, longitudinal diagnostic information such as a counter indicating a number of health events or an average duration per health event no longer provides an accurate representation of patient health.

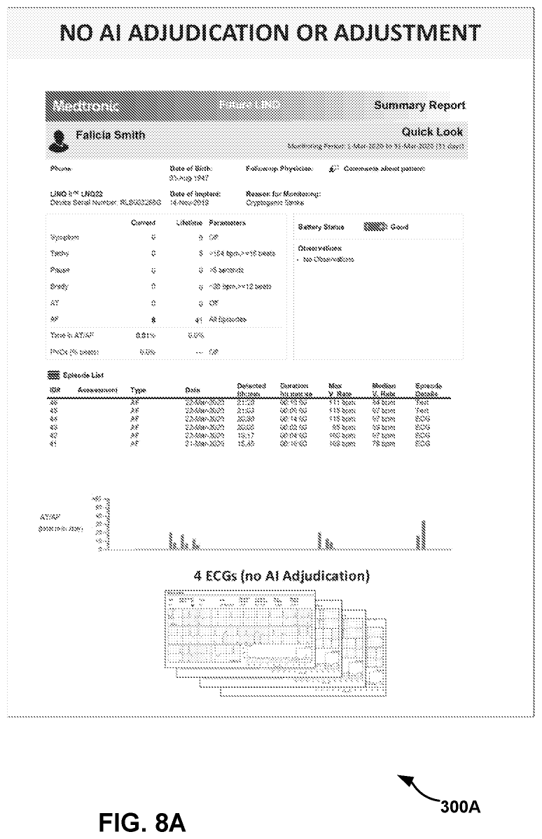

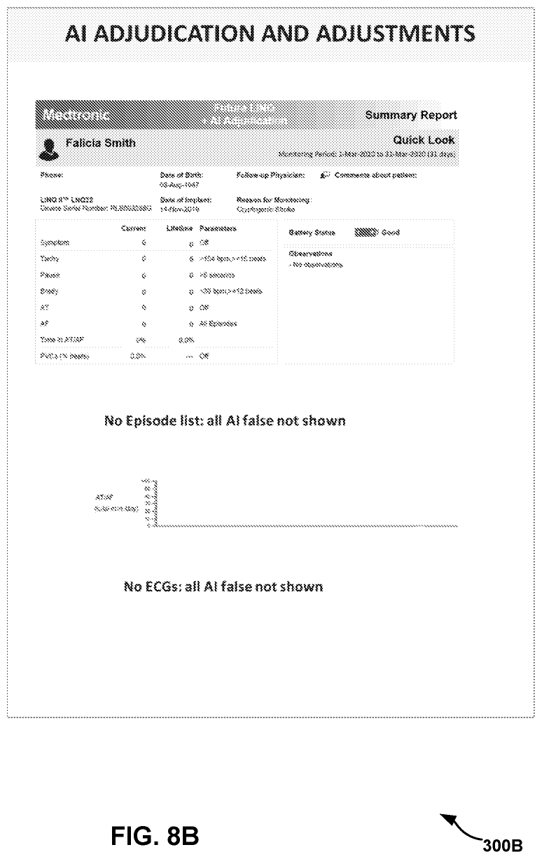

The example operation illustrated in FIG. 6 allows for removal of inaccurate health event data and retaining of accurate health event data without requiring more memory resources on IMD 10 to store additional samples of related patient data. By adjusting the device history, the example operation enables monitoring service 6 to provide an adjusted device history that more accurately reflects changes in patient health. Monitoring service 6 may perform the example operation to benefit a clinician's review of the related patient health. As depicted in FIG. 8B, the generated output allows a clinician to focus their review on accurate patient data. The example operation also allows monitoring service 6 to avoid adjudicating more samples. Otherwise, monitoring service 6 would have to adjudicate significantly more (if not all) initial detections of health events to achieve a same level of accuracy.

In the example operation of FIG. 6, the processing circuitry within the server computing device operated by monitoring service 6 determines whether any of the samples of patient data is not adjudicated (i.e., unqualified health event(s)) (110). Monitoring service 6 may set forth a number of reasons for a sample of patient data to avoid adjudication. For any one reason, a sample of patient data may not qualify for adjudication and thus, may be disqualified by the processing circuitry. IMD 10, by enforcing memory restrictions, may store a limited subset of the patient data collected during the first time period. For each sample stored in IMD 10 and/or transmitted to monitoring service 6, IMD 10 and/or monitoring service 6 may determine whether that patient data sample qualifies for adjudication. If the patient data sample is disqualified for some reason, IMD 10 and/or monitoring service 6 may bypass an application of adjudication logic.

Based on determining that the health event data includes at least one non-adjudicated sample of patient data (YES of 110), the processing circuitry within the server computing device operated by monitoring service 6 proceeds to retain the health event data corresponding to the non-adjudicated health events (140). As an option, monitoring service 6 may also retain the health event data corresponding to any adjudicated health event determined to a true detection. Based on determining that the health event data includes at least one non-adjudicated sample of patient data (NO of 110), the processing circuitry within the server computing device operated by monitoring service 6 proceeds to a determination as to whether at least one adjudicated sample of patient data is a true positive (120).

Based on determining that at least one adjudicated sample of patient data is a true positive (YES of 120), the processing circuitry within the server computing device operated by monitoring service 6 proceeds to retain a portion of the health event data corresponding to the non-adjudicated health events and any true positive(s) amongst the adjudicated health events (140). In some examples, the processing circuitry may adjudicate a sample of patient data as a false positive and then, remove any health event data corresponding to that adjudicated health event while retaining the above portion of the health event data. Based on removing the health event data corresponding to the sample that has been adjudicated as the false positive, the processing circuitry may adjust any related longitudinal diagnostic information of a second and longer time period. Considering an example in which the health event data includes five samples of patient data and only two samples were adjudicated as true positives, the processing circuitry decrements a health event counter (e.g., an Atrial Fibrillation counter) only by a number of adjudicated false positives (i.e., three (3)).

Although only a fraction of the adjudicated samples of patient data were false positives, the processing circuitry within the server computing device operated by monitoring service 6 may identify one or more non-adjudicated samples as false positives and remove any corresponding health event data. The health event data in the examples described for FIG. 6 may cover the first time period and be arranged into a data structure where each detected health event is an entry consisting of multiple data elements. In each entry, the data elements include characteristics of the health event of which some characteristics may be used to predict whether the health event is a false positive or a true positive without any sample of patient data to adjudicate. To illustrate, an example entry for any given health event may store characteristics identifying a heartrate during onset (of the health event), an activity during onset (e.g., based on accelerometer data), a respiration rate during onset, an impedance level during onset, a fluid level, an optical heartrate, heart sound measurement, and/or the like. If a substantial number of these characteristics match corresponding characteristics of the above-mentioned adjudicated samples of false positives, the processing circuitry may determine that the non-adjudicated health event also is a false positive. As an option, the processing circuitry may program IMD 10 to expand the number of data elements recorded for each health event.

Having the above-mentioned characteristics for each health event allows the processing circuitry within the server computing device operated by monitoring service 6 to adjust other trends or estimates (e.g., in the longitudinal diagnostic information described herein) for the patient. Given a sample of patient data that has been adjudicated to be a false positive, the processing circuitry may apply a machine learning model to a snippet of a respiration signal (e.g., a respiration rate) or a snippet of an impedance signal (e.g., an impedance measurement) for the same false positive and adjust any information derived from the respiration rate or the impedance measurement. The adjusted information may cover a first time period (e.g., one day). The adjusted information may be used to retabulate the other trends and estimates for a second time period (e.g., one week) that includes the first time period. As one example, the processing circuitry may use the adjusted information to adjust a heart rate variability (HRV) for the first time period (e.g., one day) or the second time period (e.g., one week). As another example, a fluid index (e.g., a heart fluid rate) may be another trend or estimate that can be adjusted based on the characteristics of the sample adjudicated to a false positive.

If the processing circuitry uses the characteristics of the above sample to identify a non-adjudicated health event that also is a false positive, the processing circuitry may remove the identified health event. Based on the removed health event, the processing circuitry may adjust certain diagnostic information for the second time period, such as the respiration rate. Similarly, the processing circuitry may use the removed health event to adjust a premature ventricular contractions (PVC) burden defined for IMD 10. The processing circuitry may compute an adjusted total number of PVCs for the second time period and/or an adjusted total number of beats. Given that PVC burden may be defined as a certain percent of a total number of PVCs divided by a total number of beats, the processing circuitry may compute an adjusted PVC burden based on the adjusted total number of PVCs and/or the adjusted total number of beats. In some examples, the processing circuitry may use return the adjusted diagnostic information to IMD 10 where corresponding trends and/or estimates are adjusted accordingly.

It should be noted that there are a number of possible alternatives for the criterion described for decision block 120; the step for determining that there is at least one true positive amongst the adjudicated samples of patient data is one example, and other examples may require at least N true positives where N is an integer greater than 1, a fraction, or a percentage of the (total) number of adjudicated samples. Based on determining that the health event data includes at least one adjudicated sample of patient data that is a true positive (NO of 120), the processing circuitry within the server computing device operated by monitoring service 6 proceeds to a determination as to whether all the adjudicated samples of patient data are false positives (130).

Alternatively, the processing circuitry within the server computing device operated by monitoring service 6 may compute a confidence value for the non-adjudicated health events and then, determine whether the confidence value of the adjudicated health events exceeds a threshold. The confidence value may indicate a likelihood that the non-adjudicated health events were true detections. If the processing circuitry determines that the confidence value fails to satisfy the threshold, the processing circuitry may treat the non-adjudicated health events as false detections and remove them from the health event data.

Based on determining that all the adjudicated samples of patient data are false positives (YES of 130), the processing circuitry within the server computing device operated by monitoring service 6 proceeds to delete both the health event data corresponding to the non-adjudicated health events and the health event data corresponding to the adjudicated health events (150). Based on determining that not all the adjudicated samples of patient data are false positives (NO of 130), the processing circuitry within the server computing device operated by monitoring service 6 retains a portion of the health event data corresponding to the non-adjudicated health events while removing a different portion of the health event data corresponding to the adjudicated health events (140).

As an alternative to determining whether all the adjudicated samples of patient data are false positives, the processing circuitry may delete both the health event data corresponding to the non-adjudicated health events and the health event data corresponding to the adjudicated health events if a health event with a longest duration is adjudicated as a false positive. There may be a sample of patient data associated with the longest health event or the duration may be indicated in a respective entry of the database record for the first time period (e.g., a day). As described herein, each health event may have an entry in the database record listing a number of attributes (e.g., a duration, an event type, and/or the like).

As an option, the processing circuitry may program a variable time period for adjudication/adjustment into the service computing device operated by monitoring service 6 and/or IMD 10. The processing circuitry may adjust the time period into an adjusted time period for a next application of the at least one criterion.

The order and flow of the operation illustrated in FIG. 6 is an example. In other examples according to this disclosure, more or fewer criterion may be considered. Further, in some examples, processing circuitry may perform or not perform the method of FIG. 6, or any of the techniques described herein, as directed by a user, e.g., via external device 12 or computing devices 99. For example, a patient, clinician, or other user may turn on or off functionality for identifying changes in patient health (e.g., using Wi-Fi or cellular services) or locally (e.g., using an application provided on a patient's cellular phone or using a medical device programmer).

FIG. 7 is a block diagram illustrating an example flow via logic 200 to provide accurate health event data for clinician review after adjudication and adjustment, in accordance with one or more examples of the present disclosure.

As illustrated, logic 200 may be partitioned into logical components for performing different functionalities. One component, adjudication logic (illustrated in FIG. 7 as “AI”), may be scheduled for or triggered into execution as directed by a medical device, a computing service, or another device, such as a mobile device, for one or more samples of patient data. The adjudication logic may be configured to verify results from an initial detection analysis in the medical device (illustrated in FIG. 7 as “ICM” for representing an implanted cardiac monitor). Given a sample of patient data purported to be a possible health event, the adjudication logic may confirm the sample is a true detection of a health event or reject the same as a false detection.

Another component, adjustment logic (illustrated in FIG. 7 as “Adjustment Logic”), may be invoked after execution of the adjudication logic. As described herein, the adjustment logic may be configured to determine whether health event data corresponding non-adjudicated health events is reliable based on results from the adjudication logic.

In the example flow of FIG. 7, the adjustment logic of logic 200 may be triggered and/or scheduled for execution between intervals of a fixed (e.g., one (1) day) or variable time period (e.g., one or more days). During the interval of the time period, the ICM may perform an initial detection analysis to determine whether any one or more of a sequence of patient data samples represents a true arrhythmia. A given sample either provides sufficient evidence for the true arrhythmia (according to the initial detection analysis) or is non-actionable (i.e., false), most likely representing a non-cardiac event. At an end of the time period, the ICM may generate health event data identifying potential occurrences of true arrhythmias. At a later point-in-time, the ICM may upload, to a cloud computing service (e.g., monitoring service 6) for adjudication, the health event data and sampled patient data for respective ones of at least a portion of the possible occurrences of true arrythmias.

In response to the adjudication logic identifying one or more samples that are false detections of cardiac events, the adjustment logic may delete the one or more false detections from the health event data and then, remove any indicia of the deleted health event data from other data for inaccurately representing patient cardiac health. By “indicia”, the present disclosure may refer to longitudinal diagnostic information (e.g., a counter, a duration, and/or the like) pertaining to ICM history with the patient. This leaves in the remaining health event data a number of non-adjudicated cardiac events. Deleting the health event data for the non-adjudicated cardiac events and removing corresponding longitudinal diagnostic information may depend on one or more criterion. For instance, if at least one adjudicated cardiac event is a true detection, the adjustment logic may determine that the health event data is most likely accurate and is to be retained on the cloud computing service. the adjustment logic may designate the health event data for permanent storage. If all the adjudicated cardiac events are false detections, the adjustment logic proceeds to remove any data incorporating the adjudicated cardiac events as if these events are true detections.

FIGS. 8A-8B are each a representation of example output for clinician review from the example flow of FIG. 7, in accordance with one or more examples of the present disclosure. Example output 300A of FIG. 8A may generated by logic (e.g., logic 200 of FIG. 7) in which adjudication logic is bypassed. Example output 300B of FIG. 8B may generated by the same logic (e.g., logic 200 of FIG. 7) in which adjudication logic and adjustment logic are executed.

When compared to each other, example output 300A of FIG. 8A includes a number of inaccuracies that are absent from example output 300B of FIG. 8B. Example output 300A of FIG. 8A presents a device history incorporating a number of health events that have been identified in health event data from a current time period. Clearly, some (if not all) the identified health events are false detections. As part of that device history, example output 300A of FIG. 8A further provides examples of longitudinal diagnostic information for a lifetime of the medical device. These examples are rendered inaccurate based on the above inaccuracies.

To identify these inaccuracies, a computing device may execute the adjudication logic to generate data indicating that one or more health events are most likely false detections. The computing device may execute the adjustment logic to determine that the non-adjudicated health events most likely are also false detections. As a result, the computing device may generate example output 300B of FIG. 8B to present a device history where these false detections have been removed from the health event data.

In a clinical or hospital setting, example output 300A of FIG. 8A provides a clinician with an inaccurate picture of a patient's health. Given that the clinician has authority of the patient's medical care, example output 300A may cause a wrong diagnosis or treatment. Example output 300B resolves the inaccurate picture and provide a device history reflecting a more accurate picture of the patient's health.

Example 1. A medical system comprising: an implantable medical device comprising: sensing circuitry configured to sense patient activity including one or more of a cardiac electrical signal, impedance, or motion of a patient; communication circuitry configured to communicate with a remote computing device; and processing circuitry configured to: analyze the sensed patient activity; detect health events of the patient based on the analysis; and transmit health event data to the remote computing device for the detected health events; and the remote computing device comprising: processing circuitry; and memory comprising programming instructions that, when executed by the processing circuitry, cause the processing circuitry to: store the health event data received from the implantable medical device in a record for the patient; apply at least one criterion to the health event data stored in the record for the patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, remove the health event data corresponding to the adjudicated health events and the non-adjudicated health events from the record; adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

Example 2. The medical system of Example 1, wherein the instructions cause the processing circuitry to apply the at least one criterion to the adjudicated health events.

Example 3. The medical system of any of Examples 1-2, wherein to remove the health event data, the instructions cause the processing circuitry to: determine that the adjudicated health events satisfy the at least one criterion.

Example 4. The medical system of any of Examples 1-3, wherein to remove the health event data, the instructions cause the processing circuitry to: determine that none of the adjudicated health events was adjudicated a true detection of the health event.

Example 5. The medical system of any of Examples 1-4, wherein to remove the health event data, the instructions cause the processing circuitry to: determine that each of the adjudicated health events was adjudicated a false detection of the health event.

Example 6. The medical system of any of Examples 1-5, wherein to remove the health event data, the instructions cause the processing circuitry to: determine that the health event data does not include a health event that failed to qualify for adjudication.

Example 7. The medical system of any of Examples 1-6, wherein to remove the health event data, the instructions cause the processing circuitry to: determine that a confidence value of the non-adjudicated health events falls below a threshold.

Example 8. The medical system of any of Examples 1-7, wherein to remove the health event data, the instructions cause the processing circuitry to: adjusting the time period into an adjusted time period for a next application of the at least one criterion.

Example 9. The medical system of any of Examples 1-8, wherein the processing circuitry of the implantable medical device collects the health event data in response to detecting the plurality of health events during the first time period.

Example 10. The medical system of any of Examples 1-9, wherein the computing device receives the health event data from the implanted medical device via a network.

Example 11. The medical system of any of Examples 1-10, wherein the implantable medical device comprises at least one of a pacemaker/defibrillator or a ventricular assist device (VAD) that comprises one or more sensors and sensing circuitry.

Example 12. The medical system of any of Examples 1-11, wherein the implantable medical device comprises an insertable cardiac monitor.

Example 13. The medical system of any of Examples 1-12, wherein the implantable medical device is configured to capture, for the health event data, samples of sensed patient activity corresponding to detection of the health events within the time period.

Example 14. The medical system of any of Examples 1-13, wherein the one or more adjudicated health events comprise the samples of sensed patient activity and the one or more non-adjudicated health events do not comprise the samples of sensed patient activity.

Example 15. The medical system of any of Examples 1-14, wherein the health events comprise initial detections of arrhythmias.

Example 16. The medical system of any of Examples 1-15, wherein the processing circuitry of the computing device is configured to adjudicate the adjudicated health events by applying a machine learned model to the health event data of the adjudicated health events.

Example 17. A method performed by a medical system, comprising: applying, by a remote computing device of the medical system, at least one criterion to health event data stored in a record for a patient for determining whether to remove at least a portion of the health event data from the record, wherein an implanted medical device of the patient communicates the health event data to the remote computing device, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, remove the health event data corresponding to the adjudicated health events and the non-adjudicated health events from the record; adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

Example 18. The method of Example 17, wherein a first structure of the health event data comprises metadata for identifying any one of the non-adjudicated health events and a second structure of the health event data comprises for any one of the adjudicated events, metadata for identifying that adjudicated health event and a sample of sensed patient data.

Example 19. The method of any of Examples 17-18, wherein a third structure of the health event data comprises for any one of a number of unqualified health events, metadata for identifying that unqualified health event, a sample of sensed patient data captured during that unqualified health event, and a reason for failing to qualify for adjudication.

Example 20. The method of any of Examples 17-19, wherein applying the at least one criterion comprises applying the at least one criterion to the adjudicated health events.

Example 21. The method of any of Examples 17-20, wherein removing the health event data comprises removing the health event data based on determining that the adjudicated health events satisfy the at least one criterion.

Example 22. The method of any of Examples 17-21, wherein removing the health event data comprises removing the health event data based on determining that that none of the adjudicated health events was adjudicated a true detection of the health event.

Example 23. The method of any of Examples 17-22, wherein removing the health event data comprises removing the health event data based on determining that each of the adjudicated health events was adjudicated a false detection of the health event.

Example 24. The method of any of Examples 17-23, wherein removing the health event data comprises removing the health event data based on determining that the health event data does not include a health event that failed to qualify for adjudication.

Example 25. The method of any of Examples 17-24, wherein removing the health event data comprises removing the health event data based on determining that a confidence value of the non-adjudicated health events falls below a threshold.

Example 26. The method of any of Examples 17-25, further comprising adjusting the time period into an adjusted time period for a next application of the at least one criterion based on removing the health event data.

Example 27. The method of any of Examples 17-26, wherein the implantable medical device collects the health event data in response to detecting the plurality of health events during the first time period.

Example 28. The method of any of Examples 17-27, further comprising receiving the health event data by the remote computing device from the implanted medical device via a network.

Example 29. The method of any of Examples 17-28, wherein the implanted medical device comprises at least one of a pacemaker/defibrillator or a ventricular assist device (VAD) that comprises one or more sensors and sensing circuitry.

Example 30. The method of any of Examples 17-29, wherein the implanted medical device comprises an insertable cardiac monitor.

Example 31. The method of any of Examples 17-30, wherein the health event data comprises samples of sensed patient activity captured by the implanted medical device corresponding to detection of the health events within the time period.

Example 32. The method of any of Examples 17-31, wherein the one or more adjudicated health events comprise the samples of sensed patient activity and the one or more non-adjudicated health events do not comprise the samples of sensed patient activity.

Example 33. The method of any of Examples 17-32, wherein the health events comprise initial detections of arrhythmias by the implanted medical device.

Example 34. The method of any of Examples 17-33, further comprising adjudicating the adjudicated health events by applying a machine learned model to the health event data of the adjudicated health events.

Example 35. A non-transitory computer readable storage medium comprising program instructions configured to cause processing circuitry of a remote computing device of a medical system to perform steps of the method of any of Examples 17-34 comprising: applying at least one criterion to health event data stored in a record for a patient for determining whether to remove at least a portion of the health event data from the record, wherein an implanted medical device communicates the health event data to a remote device of the medical system, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, removing the health event data corresponding to the adjudicated health events and the non-adjudicated health events from the record; adjusting longitudinal diagnostic information of a second time period that includes the first time period based on removing the adjudicated health events and the non-adjudicated health events from longitudinal diagnostic information of the time period; and generating output data indicative of the modified longitudinal diagnostic information of the second time period.

Example 36. A medical system comprising: a first device comprising: sensing circuitry configured to sense patient activity including one or more of a cardiac electrical signal, impedance, or motion of a patient; communication circuitry configured to communicate with a second, remote device; and processing circuitry configured to: analyze the sensed patient activity; detect health events of the patient based on the analysis; and transmit health event data to the second device for the detected health events; and the second device comprising: processing circuitry; and memory comprising programming instructions that, when executed by the processing circuitry, cause the processing circuitry to: store the health event data received from the implantable medical device in a record for the patient, and apply at least one criterion to health event data stored in a record for a patient for determining whether to remove at least a portion of the health event data from the record, wherein a remote device of the medical system receives the health event data from the implanted medical device, wherein the health event data comprises one or more adjudicated health events and one or more non-adjudicated health events over a first time period; and means for removing the health event data corresponding to the adjudicated health events and the non-adjudicated health events from the record based on a determination that the health event data satisfies the at least one criterion, wherein the instructions further cause the processing circuitry to: adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

Example 37. A medical system comprising: processing circuitry; and memory comprising programming instructions that, when executed by the processing circuitry, cause the processing circuitry to: apply at least one criterion to the health event data stored in a record for a patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event; based on a determination that the health event data satisfies the at least one criterion, remove the health event data corresponding to the adjudicated health events and the non-adjudicated health events from the record; adjust longitudinal diagnostic information of a second time period that includes the first time period based on removing the adjudicated health events and the non-adjudicated health events from longitudinal diagnostic information of the time period; and generate output data indicative of the modified longitudinal diagnostic information of the second time period.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A medical system comprising:
an implantable medical device comprising:
sensing circuitry configured to sense patient activity including one or more of a cardiac electrical signal, impedance, or motion of a patient;
communication circuitry configured to communicate with a remote computing device; and
processing circuitry configured to:
analyze the sensed patient activity;
detect health events of the patient based on the analysis; and
transmit health event data to the remote computing device for the detected health events; and
the remote computing device comprising:
processing circuitry; and
a memory comprising programming instructions that, when executed by the processing circuitry, cause the processing circuitry to:
store the health event data received from the implantable medical device in a record for the patient;
apply at least one criterion to the health event data stored in the record for the patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event;
based on a determination that the health event data satisfies the at least one criterion, remove the health event data corresponding to the adjudicated health events and the non-adjudicated health events from the record;
adjust longitudinal diagnostic information of a second time period that includes the first time period based on removing the adjudicated health events and the non-adjudicated health events from longitudinal diagnostic information of the time period; and
generate output data indicative of the modified longitudinal diagnostic information of the second time period.

2. The medical system of claim 1, wherein the instructions cause the processing circuitry to apply the at least one criterion to the adjudicated health events.

3. The medical system of claim 1, wherein to remove the health event data, the instructions cause the processing circuitry to:
determine that the adjudicated health events satisfy the at least one criterion.

4. The medical system of claim 1, wherein to remove the health event data, the instructions cause the processing circuitry to:
determine that none of the adjudicated health events was adjudicated a true detection of the health event.

5. The medical system of claim 1, wherein to remove the health event data, the instructions cause the processing circuitry to:
determine that each of the adjudicated health events was adjudicated a false detection of the health event.

6. The medical system of claim 1, wherein to remove the health event data, the instructions cause the processing circuitry to:
determine that the health event data does not include a health event that failed to qualify for adjudication.

7. The medical system of claim 1, wherein to remove the health event data, the instructions cause the processing circuitry to:
determine that a confidence value of the non-adjudicated health events falls below a threshold.

8. The medical system of claim 1, wherein to remove the health event data, the instructions cause the processing circuitry to:
adjusting the time period into an adjusted time period for a next application of the at least one criterion.

9. The medical system of claim 1, wherein the processing circuitry of the implantable medical device collects the health event data in response to detecting the plurality of health events during the first time period.

10. The medical system of claim 1, wherein the computing device receives the health event data from the implanted medical device via a network.

11. The medical system of claim 1, wherein the implantable medical device comprises at least one of a pacemaker/defibrillator or a ventricular assist device (VAD) that comprises one or more sensors and sensing circuitry.

12. A method performed by a medical system, comprising:
applying, by a remote computing device of the medical system, at least one criterion to health event data stored in a record for a patient for determining whether to remove at least a portion of the health event data from the record, wherein an implanted medical device of the patient communicates the health event data to the remote computing device, wherein the health event data comprises one or more adjudicated health events and one or more non-adjudicated health events over a first time period, and wherein the one or more adjudicated events are adjudicated as true detections of the health event or false detections of the health event;
based on a determination that the health event data satisfies the at least one criterion, remove the health event data corresponding to the adjudicated health events and the non-adjudicated health events from the record;
adjust longitudinal diagnostic information of a second time period that includes the first time period based on removing the adjudicated health events and the non-adjudicated health events from longitudinal diagnostic information of the time period; and
generate output data indicative of the modified longitudinal diagnostic information of the second time period.

13. A non-transitory computer readable storage medium comprising program instructions configured to cause processing circuitry of a remote computing device of a medical system to perform:
applying at least one criterion to health event data stored in a record for a patient for determining whether to remove at least a portion of the health event data from the record, wherein an implanted medical device communicates the health event data to a remote device of the medical system, wherein the health event data comprises one or more adjudicated health events and one or more non-adjudicated health events over a first time period, and wherein the one or more adjudicated events are adjudicated as true detections of the health event or false detections of the health event;
based on a determination that the health event data satisfies the at least one criterion, removing the health event data corresponding to the adjudicated health events and the non-adjudicated health events from the record;
adjusting longitudinal diagnostic information of a second time period that includes the first time period based on removing the adjudicated health events and the non-adjudicated health events from longitudinal diagnostic information of the time period; and
generating output data indicative of the modified longitudinal diagnostic information of the second time period.

14. A medical system comprising:
a first device comprising:
sensing circuitry configured to sense patient activity including one or more of a cardiac electrical signal, impedance, or motion of a patient;
communication circuitry configured to communicate with a second, remote device; and
processing circuitry configured to:
analyze the sensed patient activity;
detect health events of the patient based on the analysis; and
transmit health event data to the second device for the detected health events; and
the second device comprising:
processing circuitry; and
memory comprising programming instructions that, when executed by the processing circuitry, cause the processing circuitry to:
store the health event data received from the implantable medical device in a record for the patient, and apply at least one criterion to health event data stored in a record for a patient for determining whether to remove at least a portion of the health event data from the record, wherein a remote device of the medical system receives the health event data from the implanted medical device, wherein the health event data comprises one or more adjudicated health events and one or more non-adjudicated health events over a first time period; and
means for removing the health event data corresponding to the adjudicated health events and the non-adjudicated health events from the record based on a determination that the health event data satisfies the at least one criterion, wherein the instructions further cause the processing circuitry to:
adjust longitudinal diagnostic information of a second time period that includes the first time period based on removing the adjudicated health events and the non-adjudicated health events from longitudinal diagnostic information of the time period; and
generate output data indicative of the modified longitudinal diagnostic information of the second time period.

15. A medical system comprising:
processing circuitry; and
memory comprising programming instructions that, when executed by the processing circuitry, cause the processing circuitry to:
apply at least one criterion to the health event data stored in a record for a patient for determining whether to remove at least a portion of the health event data from the record, wherein the health event data comprises one or more adjudicated health events and one or more non-adjudicated health events over a first time period, and wherein the one or more adjudicated events are adjudicated as true detections of the health event or false detections of the health event;
based on a determination that the health event data satisfies the at least one criterion, remove the health event data corresponding to the adjudicated health events and the non-adjudicated health events from the record;
adjust longitudinal diagnostic information of a second time period that includes the first time period based on removing the adjudicated health events and the non-adjudicated health events from longitudinal diagnostic information of the time period; and
generate output data indicative of the modified longitudinal diagnostic information of the second time period.

16. The method of claim 12, wherein a first structure of the health event data comprises metadata for identifying any one of the non-adjudicated health events and a second structure of the health event data comprises for any one of the adjudicated events, metadata for identifying that adjudicated health event and a sample of sensed patient data.

17. The method of claim 12, wherein a third structure of the health event data comprises for any one of a number of unqualified health events, metadata for identifying that unqualified health event, a sample of sensed patient data captured during that unqualified health event, and a reason for failing to qualify for adjudication.

18. The method of claim 12, wherein applying the at least one criterion comprises applying the at least one criterion to the adjudicated health events.

19. The method of claim 12, wherein removing the health event data comprises removing the health event data based on determining that the adjudicated health events satisfy the at least one criterion.

20. The method of claim 12, further comprising adjusting the time period into an adjusted time period for a next application of the at least one criterion based on removing the health event data.
